(19) United States
(12) Patent Application Publication
(10) Pub. No.: US 2010/0114315 A1
(43) Pub. Date: May 6, 2010

(54) Intramedullary locked compression screw for stabilization and union of complex ankle and subtalar deformities

(76) Inventor: Easton L. Manderson, Ashton, MD (US)

Correspondence Address: Curtis D. Kinghorn;Sirius Strategic, LLC, 6769 W. Shadow Lake Dr., Lino Lakes, MN, 55014, US

(21) Appl. No.: 12/290,563

(22) Filed: Oct. 31, 2008

(51) Int. Cl. A61F 2/28; A61B 17/04

(52) U.S. Cl. 623/16.11; 606/301

(57) ABSTRACT

An implant for causing fusion of bones in an ankle is disclosed. The implant, in a preferred embodiment, is a cannulated screw with threads at the leading end and threads at the trailing end having a a tibial component that interacts with the tibia, a calcaneus component that interacts with the calcaneus and a midsection extending between the tibial component and the calcaneus component. The implant is placed in a borehole formed in the tibia, talus and calcaneus and causes the tibia and talus to be moved into compressive contact with each other. As a result, the ends of the tibia and talus that have previously had the cartilage removed down to bloody bone are coapted together to allow fusion. In another embodiment of the invention, a middle threaded portion is placed between the tibial component and the calcaneus component. The middle threaded portion interacts with the talus to help add compressive force to the fusion process. The invention also includes a method for using the implant to fuse the bones of the ankle together. The method includes steps of producing an implant and then using the implant to apply compressive forces on the bones of the ankle. The invention in one embodiment also includes a method that uses images such as x-ray images preoperatively to determine the length and width of the disclosed implant or any other implant with each patient's unique anatomy will properly allow coaption of the ends of the prepared tibia and talus at the ankle joint. The implant is inserted from the bottom of the foot through a predetermined hole in the calcaneus which extends through the talus into the diaphysis of the tibia. When properly inserted and seated in the bones, the implant is locked by screws.

### BACKGROUND OF THE INVENTION

1. Field of the Invention

The present invention relates generally to methods and medical devices that fuse bones in an ankle joint and more particularly to methods and medical devices for fusing bones in a painful ankle joint to relieve pain in the joint.

2. Description of Related Art

The ankle is a complex joint that, because of its position and its role in walking, takes a lot of stress and pounding. A typical human takes about one million steps a year and the stress of each step is transmitted through the respective ankle of the foot. What is commonly referred to as the ankle is actually two joints, the subtalar joint and the true ankle joint. The ends of the bones in these joints are covered by articular cartilage.

As shown in FIGS. 1 and 2, the true ankle joint is composed of three bones: the tibia, the fibula and talus. As seen from a back, or posterior, view (FIG. 2), the tibia forms the inside, or medial, portion of the ankle, the fibula forms the lateral, or outside portion of the ankle and the talus is the bone located underneath the tibia and fibula. The true ankle joint is responsible for up and down motion of the foot.

The subtalar joint is located beneath the true ankle joint and is the second part of the ankle. The subtalar joint consists of the talus on top and the calcaneus on the bottom (FIGS. 1 and 2). The subtalar joint allows side-to-side motion of the foot.

Defective cartilage in an ankle, either as a result of injury to or from degeneration of the ankle, is often a painful condition. When the ankle joint becomes chronically painful, locking the ankle bones that form the ankle joint together, a surgical method called fusion, is commonly used to relieve pain. In the traditional surgical process, the surgeon opens the ankle joint and scrapes out the remaining cartilage between the bones that are to be fused in the ankle. In this process, besides removing just cartilage, the surgeon's scrapes the bone down to bleeding bone.

Once adjacent bones that are to be fused are scraped down to bleeding bone, the surgeon places the bleeding ends of the bones together which causes the adjacent bones to grow together into one bone. Because there is no longer a joint between the bones, there is no longer pain in the joint since the two bones that previously formed the joint and caused pain by their moving are no longer able to move with respect to each other.

There are many methods currently available to hold the bones together until they grow into each other (fuse) to become one bone. One method, a technique to repair a damaged ankle using intramedullary nails, has become popular. But, this method is not without its problems. Placing the nails requires the use of elaborate jigs to precisely locate the nails and more particularly precisely insert locking screws that are placed through small holes in the nails. The combination of the nails with the locking screws locates the nail in place in the appropriate bones of the ankle.

The requirement to locate the locking screws in the nails is a very difficult process for some surgeons. This difficulty in using intramedullary nails is unfortunate because the use of intramedullary nails allows the patient to put weight on the ankle during the ankle fusion process, a benefit that is not present in traditional fusion techniques.

Fusion of an ankle for pain due to defective cartilage with all metal or all plastic type implants such as intramedullary nails has, for the most part, given adequate pain relief. Fixation of these implants must be stable to be able to tolerate cyclical weight bearing on the implant (e.g., walking) without loosening. For this reason, the foot must be able to function in a plantigrade manner (i.e., walking on the sole with the heel touching the ground) to avoid excessive intrusive forces that may cause or accelerate loosening of the implant.

These implants, to ensure continued effective function, should not be placed in an ankle that has pain because of infection or pain caused by the presence of dead bone. The dead bone does not provide an effect anchor for such devices.

One factor may be the fact that it is difficult to learn how to effectively implant such implanted devices. Many practitioners in the art of ankle placement surgery agree that regardless of the prior art implant to be placed, as for example, intramedullary nails, there is a steep learning curve to properly perform the surgery needed to apply the implant.

A second factor may be that the ankle joint bears more weight than the hip or knee. An excessive load may doom an ankle implant device to early failure unless the design is strong and allows proper distribution of this load. It has not been easy to design ankle joint implant devices that can bear these high loads.

In view of the foregoing, there is a need for an improved ankle implant that accomplishes one of more of the following objectives: has a much less steep learning curve and is therefore easier to apply even by the general orthopaedist than traditional devices; provides a good fusion joint; is strong enough to ensure its long-term survival in a fused bone joint and provides a relatively long pain free status for a patient.

### SUMMARY OF THE INVENTION

An implant for causing fusion of bones in an ankle is disclosed. The implant, in a preferred embodiment, is a cannulated screw with threads at the leading end and threads at the trailing end having a a tibial component that interacts with the tibia, a calcaneus component that interacts with the calcaneus and a midsection extending between the tibial component and the calcaneus component. The implant is placed in a borehole formed in the tibia, talus and calcaneus and causes the tibia and talus to be moved into compressive contact with each other. As a result, the ends of the tibia and talus that have previously had the cartilage removed down to bloody bone are coapted together to allow fusion. In another embodiment of the invention, a middle threaded portion is placed between the tibial component and the calcaneus component. The middle threaded portion interacts with the bone surrounding it to help add compressive force to the fusion process between the tibea and the talus.

The invention also includes a method for using the implant to fuse the bones of the ankle together. The method includes steps of producing an implant as described herein and then using the implant to apply compressive forces on the coapted surface of the tibea and the talus.

The invention in one embodiment also includes a method called “templating” that uses images such as x-ray images preoperatively to determine the length and width of the disclosed implant or any other implant with each patient's unique anatomy to properly allow coaption of the ends of the prepared tibia and talus at the ankle joint. The implant is inserted from the bottom of the foot through a predetermined hole in the calcaneus, talus and tibia. When properly inserted and seated in the bones, the implant is typically locked by screws in the tibia and at least one screw in the calcaneus.

The double or triple threaded intramedullary ankle compression screw of the present invention is indicated for use in the very complex and difficult cases of arthropathy threatened by amputation, requiring salvage when standard approaches would be unsuitable or ineffective. The disclosed implant, as used in accordance with the methods of the invention, ensures a simpler application and a more effective function than prior art implants. It is, therefore a primary object of the present invention to provide an effective implant designed to effectuate fusion of the bones of the ankle. Other objects of this invention, in one or more embodiments, are to:

minimize bone removal around the implant;

provide an implant that is easy to use;

provide an implant that has a gentle learning curve; and

provide an implant that can endure the stress and strain of early weight bearing on an ankle undergoing fusion between the tibia and the talus.

It is therefore an object of the present invention in one or more embodiments to provide a device that meets at least one of the objects listed above. Not all of these objects need be present in a single embodiment. Instead, a particular embodiment may have one or more of these objects. These and other objects of the invention will be clear from the following detailed description of the invention in connection with the drawings.

### BRIEF DESCRIPTION OF THE DRAWINGS

The invention will be described hereafter in detail with particular reference to the drawings. Throughout this description, like elements, in whatever embodiment described, refer to common elements wherever referred to and referenced by the same reference number. The characteristics, attributes, functions, interrelations ascribed to a particular element in one location apply to that element when referred to by the same reference number in another location unless specifically stated otherwise. In addition, the exact dimensions and dimensional proportions to conform to specific force, weight, strength and similar requirements will be within the skill of the art after the following description has been read and understood.

All Figures are drawn for ease of explanation of the basic teachings of the present invention only; the extensions of the Figures with respect to number, position, relationship, and dimensions of the parts to form examples of the various embodiments will be explained or will be within the skill of the art after the following description has been read and understood.

FIG. 1 is a medial lateral view (inside side view) of the bones of the ankle.

FIG. 2 is a posterior (back) view of the bones of the ankle.

FIG. 3 is a perspective view of a preferred embodiment of the ankle fusion device.

FIG. 4 is a side view of the ankle fusion device of FIG. 3.

FIG. 5 is a side cross-sectional view of the ankle fusion device of FIG. 3 showing bone screws in place at the proximal slot and a the distal slot.

FIG. 6 is an distal end view of the ankle fusion device of FIG. 3.

FIG. 7 is an proximal end view of the ankle fusion device of FIG. 3.

FIG. 8 is a medial lateral view (inside side view) of the ankle fusion device of FIG. 3 in place in an ankle.

FIG. 9 is a plantigrade view (sole of a foot) showing the location of the ankle fusion device of FIG. 3 in the position of FIG. 8.

FIG. 10 is a side view of an alternate embodiment of the ankle fusion device.

FIG. 11 is a medial lateral view (inside side view) of the ankle fusion device of FIG. 10 in place in an ankle.

FIG. 12 is a flow chart of the “preoperative templating” process for improving the outcome of an ankle fusion procedure.

### DETAILED DESCRIPTION OF THE INVENTION

In order that the invention may be clearly understood and readily carried into effect, preferred embodiments of the invention will now be described, by way of example only and not to limit the invention, with reference to the accompanying drawings. The intramedullary ankle fusion device of the present invention is shown in the drawings generally labeled 10.

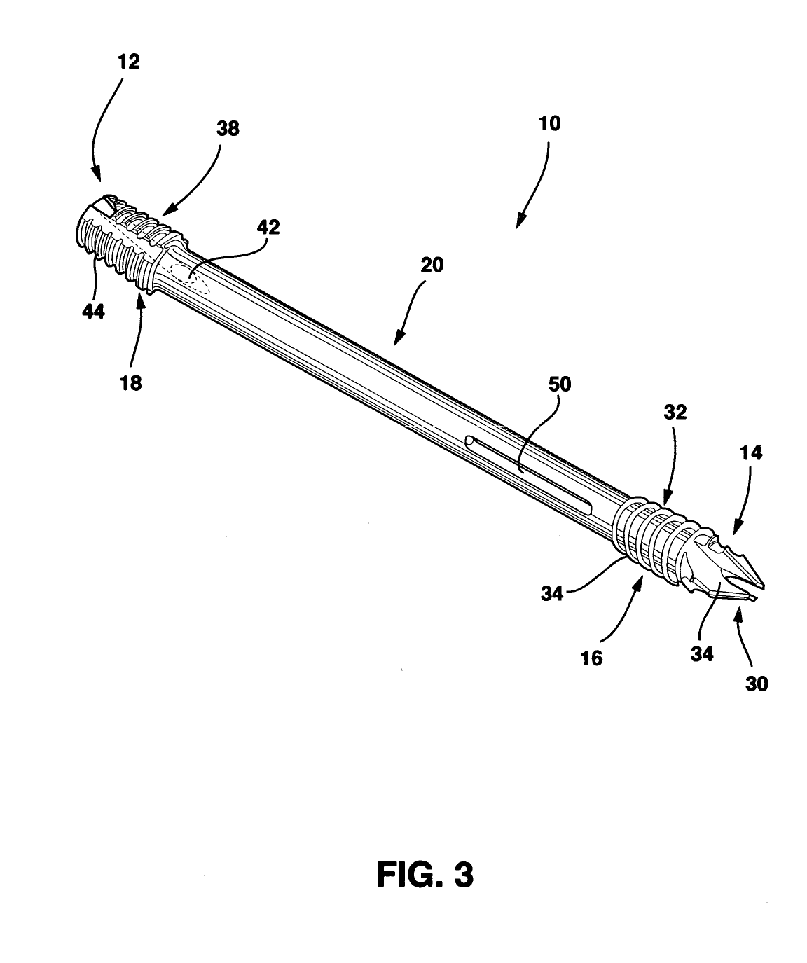

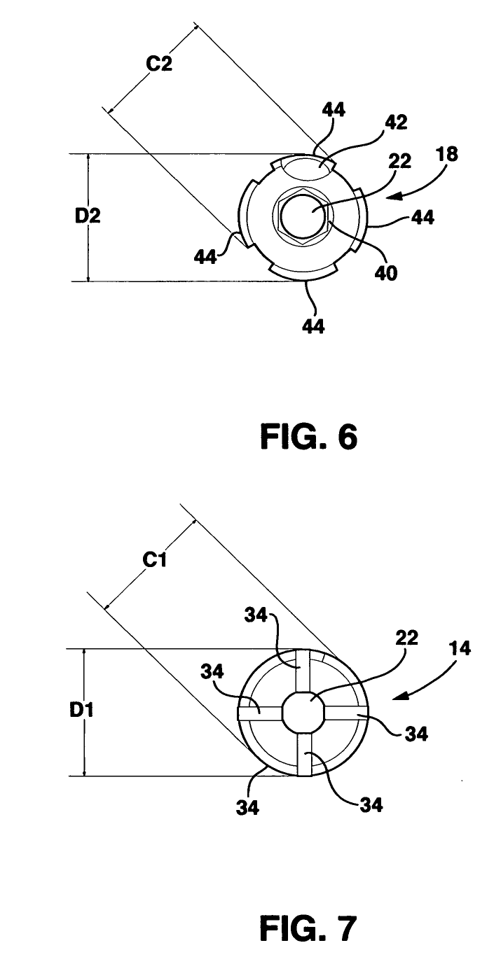

The ankle fusion device 10 is a preferred embodiment shown in FIGS. 3-8 is a double threaded cannula having a proximal end 12, an opposite distal end 14, a tibial component 16 at the distal end 14, a calcaneus component 18 at the proximal end 12 and a midsection 20 extending between the tibial component 16 and the calcaneus component 18.

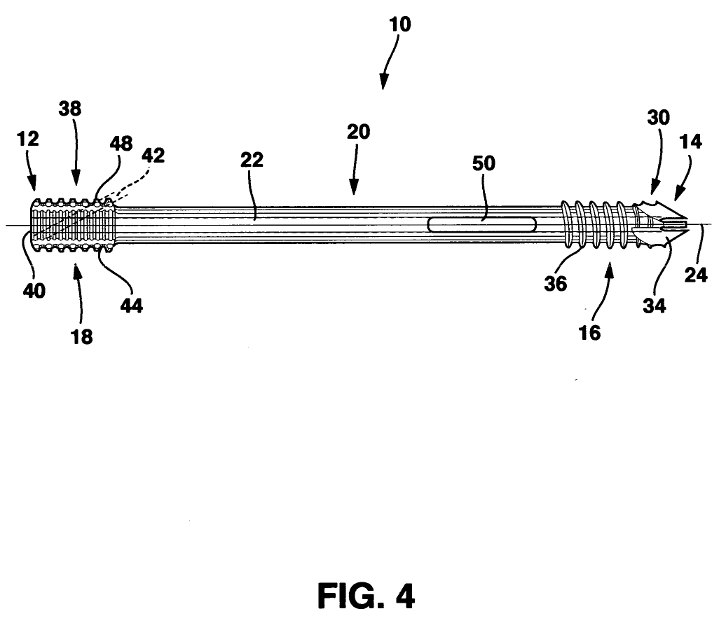

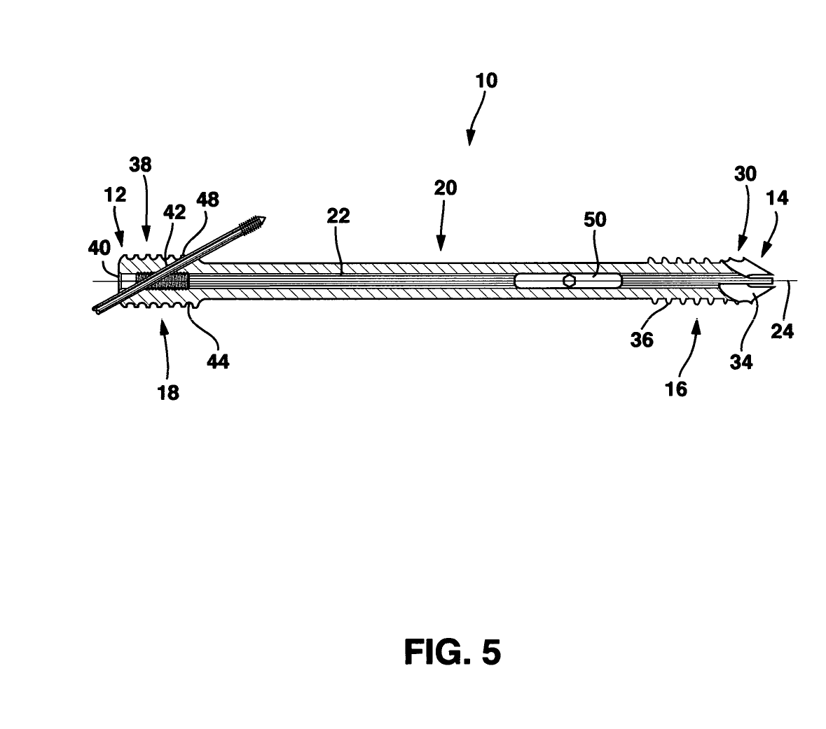

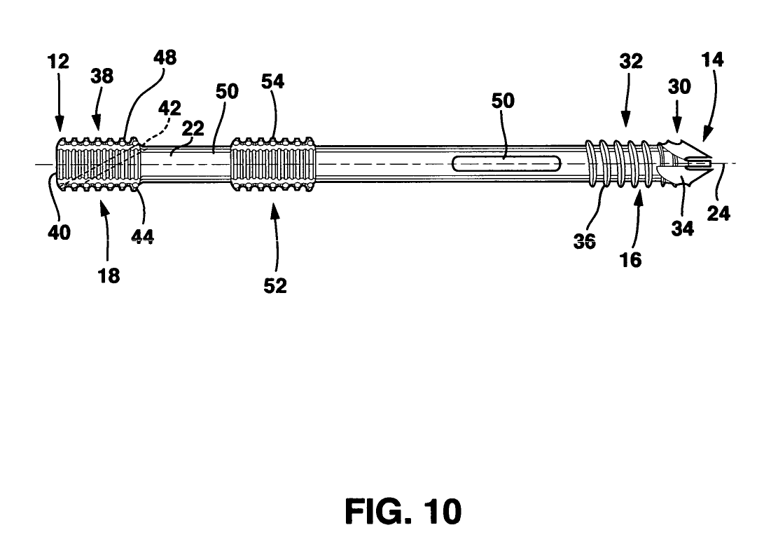

Because the ankle fusion device 10 is a cannula, the ankle fusion device 10 has a lumen 22 (FIG. 5 and shown in phantom in FIGS. 4 and 10) extending along a midline 24 of the ankle fusion device 10 from the proximal end 12 to the distal end 14.

The tibial component 16 includes a boring fixture 30 and a distal threaded portion 32. The boring fixture 30 in one embodiment preferably consists of one or more cutting blades 34 such as is common on surgical bone drills and is located on the ultimate distal end 14 of the ankle fusion device 10. For example, in one preferred embodiment, the boring fixture 30 consists of four sharpened blades 34 extending toward the distal end 14 and curving inward toward the midline 24 as the boring fixture 30 moves toward the distal end 14. The operation of the boring fixture 30 will be described later in connection with the use of the ankle fusion device 10. The function of the boring fixture 30 is to cut bone and allow the cut bone to pass into the lumen 22 to be removed from the ankle fusion device 10. In another embodiment of the ankle fusion device 10, there is the distal threaded portion 32 but no boring fixture 30. In this embodiment of the ankle fusion device 10, the function of the boring fixture is performed entirely by reamers as is described below.

The distal threaded portion 32 is located just proximal to the boring fixture 30 and is a distal screw thread 36. The distal screw thread 36 has an outer diameter “D1,” a core diameter “C1” and a constant pitch P1. The diameter D1 is the diameter of the distal screw thread 36 entirely across the distal screw thread 36. The core diameter C1 is the diameter of the distal threaded portion 32 from which the distal screw thread 36 extends. The pitch P1 is the pitch angle of the distal screw thread 36 as is well understood in the art. The function of the distal threaded portion 32 is initially to help move the ankle fusion device 10 through the borehole 28 and, in conjunction with the calcaneus component 18 as will be explained hereafter, to apply compressive force on the bones of the tibia and talus and ultimately to anchor the ankle fusion device 10 in the diaphysis portion of the tibia bone.

The calcaneus component 18 includes a proximal threaded portion 38, a tool slot 40 and a proximal slot 42. The proximal threaded portion 38 is located at the proximal end 12 and includes a proximal screw thread 44. The proximal screw thread 44 has an outer diameter “D2,” a core diameter “C2” and a constant pitch P2. The diameter D2 is the diameter of the proximal screw thread 44 entirely across the proximal screw thread 44. The core diameter C2 is the diameter of the proximal threaded portion 38 from which the proximal screw thread 44 extends. The pitch P2 is the pitch angle of the proximal screw thread 44 as is well understood in the art. The function of the proximal threaded portion 38 is initially to help move the ankle fusion device 10 through the borehole 28 and, in conjunction with the tibial component 16 as will be explained hereafter, to apply compressive force on the bones of the tibia and talus and ultimately to anchor the ankle fusion device 10 in the talus bone.

The outer diameter D2 of proximal screw thread 44 is the same size or larger than the diameter D1 of the distal threaded portion 32 in order to allow the proximal screw thread 44 to be able to interact with the walls of the borehole 28. The pitch P2 of the proximal screw thread 44 may be the same, less than or more than the pitch P1 of the distal screw thread 36. The reason the proximal screw thread 44 may have a different screw pitch than the distal threaded portion 32 will be explained hereafter in connection with the operation of the ankle fusion device 10.

As mentioned, the calcaneus component 18 also includes a tool slot 40. Tool slot 40 is a cavity formed in the proximal end 12 of the ankle fusion device 10. The cavity of the tool slot 40 has a shape that conforms with the external configuration of a male tool (not shown) that conformally mates with the tool slot 40. The function of the tool slot 40 is to receive the male tool and allow rotation of the male tool around the midline 24 to be transferred to the ankle fusion device 10 so that the ankle fusion device 10 will also rotate about the midline 24. In a preferred embodiment of the invention, the tool slot 40 is shaped to receive a hexagonal male tool. However, any shape or configuration may be used for the tool slot 40 such as is well understood in the art including, but not limited to, square, triangular, pentagonal or star, in order to conformally mate with any type of male tool that can be used to rotate the ankle fusion device 10.

The calcaneus component 18 also preferably includes a proximal slot 42 (FIGS. 3, 4, 5 and 10). The proximal slot 42 is a slot having a diameter preferably slightly larger than the diameter of a guide wire that is used to position the ankle fusion device 10 as will be explained hereafter. The proximal slot 42 extends from the lumen 22 at an angle outward through the proximal threaded portion 38 to the outer surface 48 of the proximal threaded portion 38. One function of the proximal slot 42 is to allow the guide wire to pass through the lumen 22 to and through the ultimate distal end 14 while at the same time not getting in the way of the male tool as it interacts with the tool slot 40 as described above. In this way, the guide wire passes into the lumen 22 through the proximal slot 42 at the proximal end 12 of the ankle fusion device 10.

The proximal slot 42 also preferably extends distally from the lumen 22 through the wall of the midsection 20 inline with the part of the proximal slot 42 that extends proximally from the lumen 22. The function of this distally extending portion of the proximal slot 42 is to allow the proximal end 12 of the ankle fusion device 10 to be anchored in the talus bone by a screw after compressive forces have been applied as will be explained hereafter.

Also as mentioned above, a midsection 20 extends between the tibial component 16 and the calcaneus component 18. The midsection 20 is essentially a tube and has a length chosen to correspond to different lengths, configurations and sizes of the bones of the ankle that are to be fused by the ankle fusion device 10. The lumen 22 also extends entirely through the midsection 20. The lumen 22 is sized to allow small pieces of bone removed by the boring fixture 30 to pass through the ankle fusion device 10 under suction from the distal end 14 to the proximal end 12 where the bone pieces may be removed. However, the lumen 22 should not have such a large diameter as to compromise the strength of the ankle fusion device 10, particularly the midsection 20.

In a preferred embodiment of the invention, the midsection 20 also contains at least one distal slot 50 that extends entirely through the midsection 20 from one side, through the lumen 22 and out the other side of the midsection 20. In a most preferred embodiment of the invention, distal slot 50 extends at approximately a right angle to the midline 24 that extends through the midsection 20. However, in alternate embodiments of the invention, distal slot 50 may be formed at an angle other than 90° to the midline 24. For example, and not intending to limit the range of angles, a distal slot 50 may be formed at 45 degrees to the midline 24. Preferably, the distal slot 50 is formed at approximately a right angle to the proximal slot 42 to facilitate placement of the locking bone screws as will be described hereafter. The ankle fusion device 10 may have more than one distal slot 50. Such additional slots 50 would be placed along the length of the midsection 20 either proximally or distally to the original distal slot 50. Where there is more than one distal slot 50, each distal slot 50 may be formed at 90 degrees to the midline 24 or one or more than one distal slot 50 may be formed at angles other than 90 degrees to the midline 24.

The tibial component 16, calcaneus component 18 and midsection 20 are preferably molded in one piece of a rugged, durable, biocompatible material such as medical grade stainless steel, nitenol or titanium. However, these components may be manufactured separately of the same or different material and joined together by means well understood in the art, including but not limited to welding, mechanical connection or adhesives, to form the ankle fusion device 10 described herein. Further, although these components have been described as being formed from specific metals, it is within the scope of the invention that these components could be made of non-metallic materials such as ceramics or plastics.

In an embodiment of the ankle fusion device 10, the surface of the midsection 20 is studded and sintered to enhance fixation to the surrounding bone. In another embodiment of the ankle fusion device 10, an osteoconductive coating is added to outer surface of the midsection 20 in addition to or in the alternative to the studded and sintered outer surface described above.

In an alternate embodiment of the ankle fusion device 10 shown in FIG. 10, an additional component, a middle threaded portion 52, is added along the midsection 20 between the tibial component 16 and the calcaneus component 18. The middle threaded portion 52 is located so that upon implant of the ankle fusion device 10, the middle threaded portion 52 will be located in the talus.

The middle threaded portion 52 includes a middle screw thread 54 with an outer diameter “D3,” a core diameter “C3” and a constant pitch P3. The diameter D3 is the diameter of the middle screw thread 54 entirely across the middle screw thread 54. The core diameter C3 is the diameter of the middle threaded portion 52 from which the middle screw thread 54 extends. The pitch P3 is the pitch angle of the middle screw thread 54 as is well understood in the art. In various embodiments of the ankle fusion device 10, the diameter D3 may be greater than, less than or equal to the diameter D1 of the distal threaded portion 32 or the diameter D2 of the proximal threaded portion 38. In addition, the pitch P3 of the middle threaded portion 52 may be greater than, less than of equal to the pitch P1 of the distal threaded portion 32 or the pitch P2 of the proximal threaded portion 38. The function of the middle threaded portion 52 is initially to help move the ankle fusion device 10 through the borehole 28 and, in conjunction with the tibial component 16 and the calcaneus component 18 as will be explained hereafter, to apply compressive force on the bones of the tibia and talus and ultimately to anchor the ankle fusion device 10 in the fused ankle bone.

Regarding the diameters D1, D2 and D3, if present, and the diameter of the midsection 20 in the embodiments of the ankle fusion device 10, these diameters should be large enough to allow the ankle fusion device 10 to bear weight without failure by breaking or by subsidence but also not so large as to require excessive bone removal which would weaken the now fused ankle joint. Also, the diameter of the midsection 20 should be large enough that it fills up the intramedullary canal and is tight fitting against the cortical bone. For example, and not intending to be limiting, a preferred diameter of the midsection 20 is between about 11 to 13 mm. Also, the overall length of the ankle fusion device 10 is such that the ankle fusion device 10 spans an ankle joint and pulls the talus bone into contact with the tibia. For example, and not intending to limit the dimensions, a preferred overall length of the ankle fusion device 10 is from about 150 mm to about 180 mm.

In any of the embodiments of the ankle fusion device 10 described above, any or all of the distal screw thread 36, proximal screw thread 44 or middle screw thread 54 may be segmented. “Segmented” means that the screw thread has a break extending either entirely or partially through the screw thread in a direction parallel to the midline 24. Segmenting allows the threads of the screw threads being segmented to clean itself of bone as the ankle fusion device 10 is rotated into the desired position in the bone.

In use, the intramedullary ankle fusion device 10 described above, is implanted as follows to fuse the bones of the ankle together. According to this method, the size and length of the ankle fusion device 10 is preferably first determined according to the templating method described below. Although this templating step is not required to be the first step, it is believed that doing this step first will improve the outcome of the surgery.

The patient is then placed prone on the operating table in the supine position. The transfibular approach may be used. Also, an anterior longitudinal midline incision is used to debride the cartilage and appose the tibio-talar cancellous surfaces. Using appropriate traction, all the bony surfaces are exposed for removal of cartilage, as described above. The cartilage is removed to the appropriate depth of subchondral bone to produce bleeding bone.

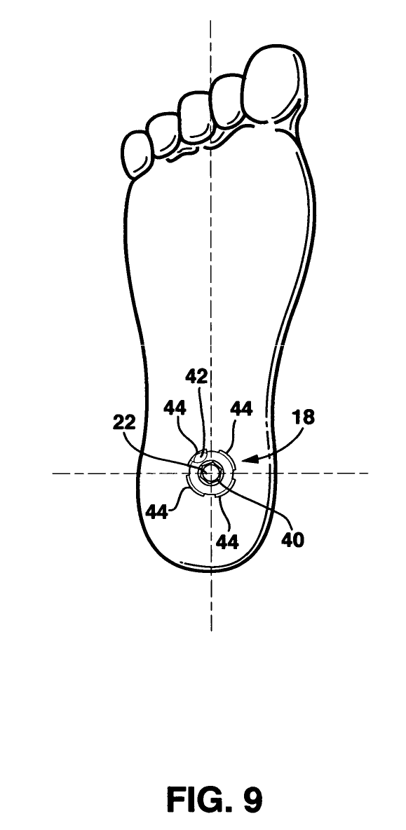

A one inch transverse incision is then made at the intersection of a line drawn along the anterior border of the fibula and proceeding along the plantar surface with a line drawn through the center of the heel or along the midline of the tibia medially as will be described below. (FIG. 9) Blunt dissection is made down to the inferior surface of the calcaneus. A periosteal elevator is used to gently push the soft tissue from the proposed entry site for a guide wire (e.g., a 3.2 mm guide wire) and the intramedullary ankle fusion device 10.

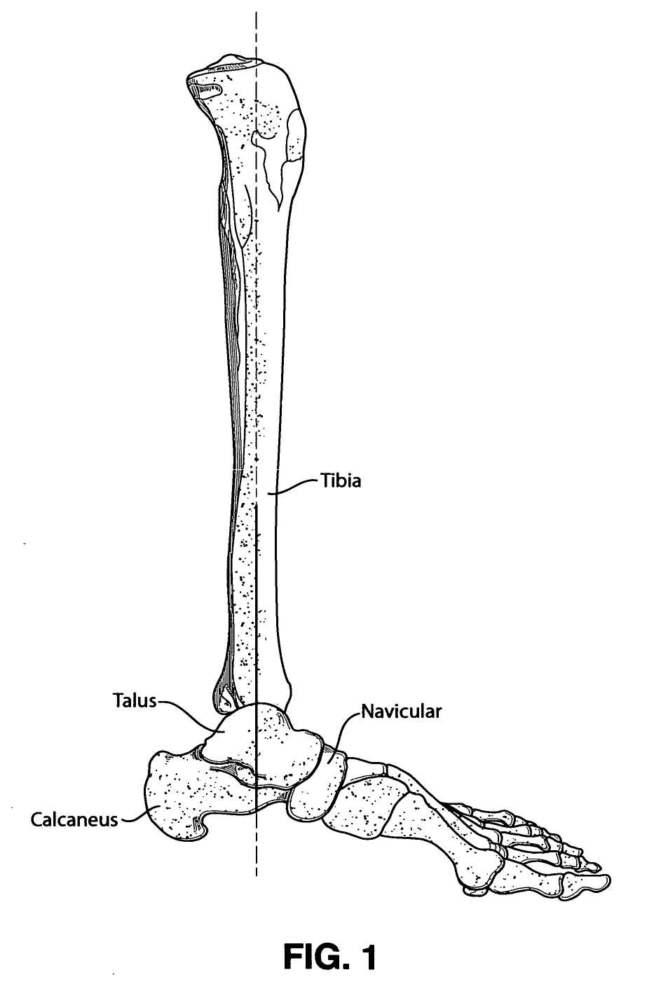

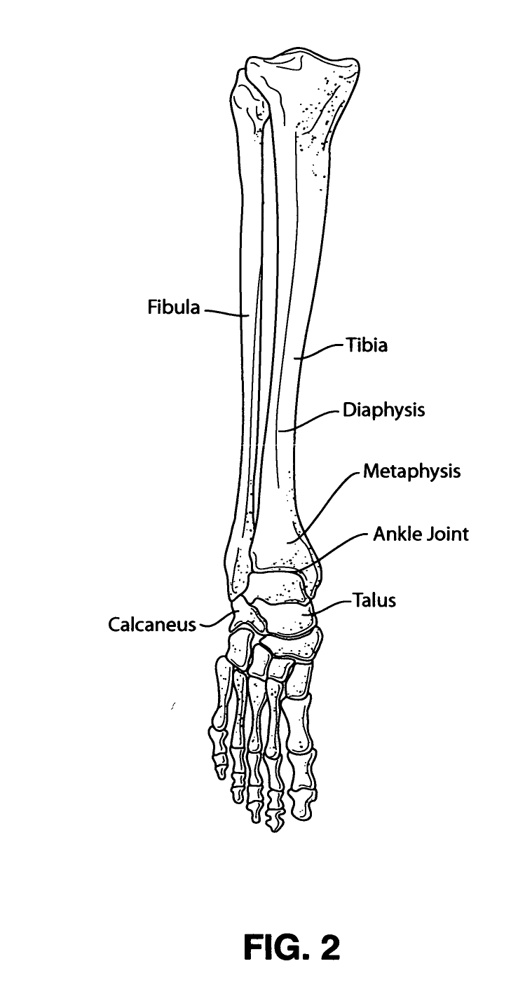

A guide hole is drilled from the sole of the heel through the calcaneus bone (heel bone) up through the ankle bones to be fused and into the tibia (FIG. 1). To locate the proper location to drill the guide hole and consequently place the guide wire, the practitioner establishes the midline of the tibia near the ankle. He or she then continues the midline downward to the sole and across the sole or plantar aspect of the foot (FIG. 9). Thereafter, a line is drawn perpendicular to this midline line through the center of the heel. The practitioner makes the one inch transverse incision at the intersection of these lines. After blunt dissection and the use of a periosteal elevator as described above the guide wire is inserted under fluoroscopic control through the calcaneus and talus and into the intramedullary canal of the distal tibia to its diaphysis. A bore hole is then drilled from the sole of the heel through the inferior surface of the calcaneus through the talus and finally into the intramedullary aspect of the distal tibia (FIG. 1).

The guide wire is inserted from the inferior surface of the calcaneus through the talus and into the intramedullary area of the tibia (FIG. 1) with the foot held in neutral position of flexion, extension, varus, valgus and rotation. The intramedullary position of the guide wire is verified by intra-operative roentgenograms or fluoroscopy and coaption and alignment confirmed.

Where the ankle fusion device 10 does not include a boring fixture 30, the ankle fusion device 10 is seated by using a relatively smaller diameter reamer (e.g., a 9 mm cannulated reamer) over the guide wire to prepare the intramedullary tibial canal and a relatively larger diameter reamer (e.g., a 13 mm canullated reamer) to prepare the calcanceal and talar canals. The bones that the respective reamers move through to form the borehole 28, moving upward from the heel bone, are the calcaneus (heel bone), ankle bone (talus), into the diaphysis of the major legbone (tibia). The guide wire precisely locates the reamers in these bones. The larger reamer should only ream to the inferior half of the talus after reaming the calcaneus.

Where the ankle fusion device 10 includes a boring fixture 30, the ankle fusion device 10 acts as a self-reaming device, at least in part. The surgeon may want to prepare a relatively smaller diameter borehole 28 using a reamer as described above and then use the boring fixture 30 to cut a larger diameter borehole 28 instead of a second separate reamer. Alternately, the boring fixture 30 may be used to entirely cut the borehole 28.

In any event, a properly sized ankle fusion device 10 is then inserted over the guide wire into this prepared borehole 28 if present or along the guide wire in the bone is there is no borehole 28 present. The guide wire is placed through the distal end 14 and through the lumen 22 so that the guide wire exits the lumen 22 at the proximal end 12 through the proximal slot 42. The size of the ankle fusion device 10, meaning the diameter and length of the ankle fusion device 10, is preferably preselected according to the templating method described below so that the proper diameter and length ankle fusion device 10 for the patient's specific anatomy is chosen. A source of vacuum (not shown) may be attached to the proximal end 12 of the ankle fusion device 10 and activated.

A male tool (not shown) is engaged with the tool slot 40 so that rotation of the male tool rotates the entire ankle fusion device 10 as the ankle fusion device 10 engages and interacts with the borehole 28. Where the ankle fusion device 10 includes a boring fixture 30, rotation of the male tool also causes the boring fixture 30 and particularly the blades 34 to rotate. As bone is removed from the borehole 28 by the blades 34 if present, the vacuum exerted at the proximal end 12 of the ankle fusion device 10 pulls any bone cut by the blades 34 through the lumen 22 and out of the ankle fusion device 10. This process continues producing a new borehole 28 having a diameter approximately equal to the diameter C1 of the distal threaded portion 32.

Regardless of how the borehole 28 is formed, at some point, the distal threaded portion 32 comes into contact with the borehole 28 formed by the reamers or the boring fixture 30. At this point, assuming rotation of the ankle fusion device 10 in the correct direction, the distal threaded portion 32 begins to cut threads into the bone of the borehole 28 and move the entire ankle fusion device 10 into and along the borehole 28.

The reason the borehole 28 has a diameter approximately equal to C1 is that C1 is the diameter of the distal threaded portion 32 from which the distal screw thread 36 extends. As a result, the distal screw thread 36 on the distal threaded portion 32 cut into the wall of the borehole 28 but do not widen the diameter of the borehole 28 so that the resulting diameter of the borehole 28 will be approximately C1 which is slightly less than a diameter D1 of the distal threaded portion 32.

In the embodiment of the ankle fusion device 10 not having a middle threaded portion 52, this process continues until the proximal threaded portion 38 comes in contact with the borehole 28. At this time, because the diameter D2 of the proximal screw thread 44 of the proximal threaded portion 38 is larger than the diameter D1 of the distal screw thread 36, the proximal screw thread 44 will begin to engage the bone forming the outer wall of the borehole 28 and will begin to cut its own threads into the bone surrounding the borehole 28.

The proximal screw thread 44 of the proximal threaded portion 38 and distal screw thread 36 of the distal threaded portion 32 will preferably have different diameters and different pitches. As a result, as the ankle fusion device 10 is rotated by engagement of the male tool with the tool slot 40, the threads of the distal screw thread 36 may want to move through the borehole 28 at a different rate than do the threads of the proximal screw thread 44.

For example, where the pitch P1 of the distal screw thread 36 is greater than the pitch P2 of the proximal screw thread 44, once the proximal screw thread 44 are engaged with the walls of the borehole 28, each rotation of the ankle fusion device 10 will then cause the distal screw thread 36 to want to move farther through the borehole 28 than will the proximal screw thread 44. As a result, rotation of the ankle fusion device 10 in this embodiment in this configuration with respect to the bone of the borehole 28 will cause the distal end 14 of the ankle fusion device 10 to pull the proximal end 12 of the ankle fusion device 10 toward it thus moving the bones of the ankle in which the distal threaded portion 32 and proximal threaded portion 38 are engaged into close and firm contact with each other thus producing the compression needed for a good fusion of the ankle bones.

As another example, in an embodiment of the ankle fusion device 10 the pitch P1 of the distal threaded portion 32 is less than the pitch P2 of the proximal threaded portion 38. In this embodiment of the ankle fusion device 10, once the proximal screw thread 44 are engaged with the walls of the borehole 28, each rotation of the ankle fusion device 10 will then cause the distal screw thread 36 to want to move less far through the borehole 28 than will the proximal screw thread 44. As a result, rotation of the ankle fusion device 10 in this embodiment in this configuration with respect to the bone of the borehole 28 will cause the proximal end 12 of the ankle fusion device 10 to push the bone it is engaged in toward the bone that the distal end 14 of the ankle fusion device 10 is engaged with. Through this process, the bones of the ankle in which the distal threaded portion 32 and proximal threaded portion 38 are engaged are moved into close and firm contact with each other thus again producing the compression needed for a good fusion of the ankle bones.

The present ankle fusion device 10 has been described herein in at least three main embodiments. In the first major embodiment, the ankle fusion device 10 is dimensioned so that the distal threaded portion 32 will be located in the intramedullary canal of the tibia and the proximal threaded portion 38 located in the talus. In the second major embodiment, the ankle fusion device 10 is dimensioned so that the distal threaded portion 32 will be located in the intramedullary canal of the tibia and the proximal threaded portion 38 located in the calcaneus. In the third major embodiment, the ankle fusion device 10 has a middle threaded portion 52 and is dimensioned so that the distal threaded portion 32 will be located in the intramedullary canal of the tibia, the middle threaded portion 52 in the talus and the proximal threaded portion 38 located in the calcaneus. In any of these embodiments, the distal threaded portion 32 must initially pass through the calcaneus on its way to being fixed in the intramedullary canal. In the second embodiment, the larger threads of the proximal threaded portion 38 will remain tightly in the calcaneus. As a result, the calcaneus is pulled to the talus and thus the talus to the tibia for coaption through compression.

The third major embodiment, with the middle threaded portion 52 ending up in the talus, is a combination of the first and second embodiments. As a result, both the proximal threaded portion 38 and the middle threaded portion 52 have about the same diameter. So, the middle threaded portion 52 and the distal threaded portion 32 work together to provide compression between the tibia and the talus. Also, the proximal threaded portion 38 and the middle threaded portion 52 work together to move the calcaneus into compression with the talus. In any of these embodiments a reamer may be used to form the borehole 28 whether for the part of the borehole 28 where the distal threaded portion 32 will ultimately be located (using a relatively small diameter reamer) or for the part of the borehole 28 where the rest of the ankle fusion device 10 will be located (using a relatively larger diameter reamer). Where a reamer is used, it is preferable but not absolutely required that the reamer be used over the guide wire. As mentioned above, in certain embodiments of the ankle fusion device 10, the boring fixture 30 may alternately cut the narrower diameter channel for the distal threaded portion 32.

In any case where a smaller diameter reamer is used, the ankle fusion device 10 is rotated until the distal end 14 contacts the smaller entrance in the tibia formed by the relatively smaller reamer. By using reamers of different diameters to create a borehole 28, the intramedullary ankle fusion device 10 automatically stops when it approaches the smaller entrance in the tibia (i.e., the 9 mm entrance). At this time, only a small portion of the proximal threaded portion 38 is left extending from the calcaneus bone. Where no reamer is used, the ankle fusion device 10 is rotated until only a small portion of the proximal threaded portion 38 is left extending from the calcaneus bone.

Once the ankle fusion device 10 is in the desired location in the borehole 28 and sufficient compressive pressure has been applied to the bones of the ankle engaged with the distal threaded portion 32 and the proximal threaded portion 38, and the middle threaded portion 52 if present, the physician can palpate the ankle fusion device 10, especially the proximal end 12 sticking out of the calcaneus bone to help locate and apply locking screws to anchor the ankle fusion device 10 in the bone. The locking screw are placed along a guide wire to allow the locking screw to follow the guide wire to ultimately be placed obliquely across the lumen 22 through the proximal slot 42 or distal slot 50 and then tightened to further lock the ankle fusion device 10 to the calcaneus or talus, respectively. Then, the guide wire is removed.

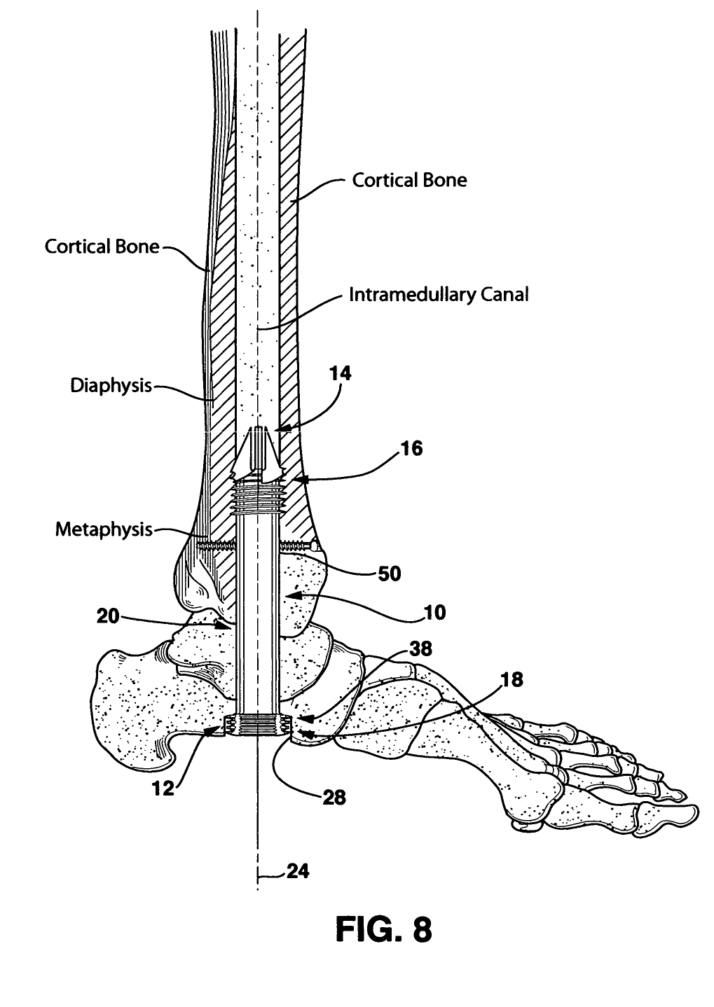

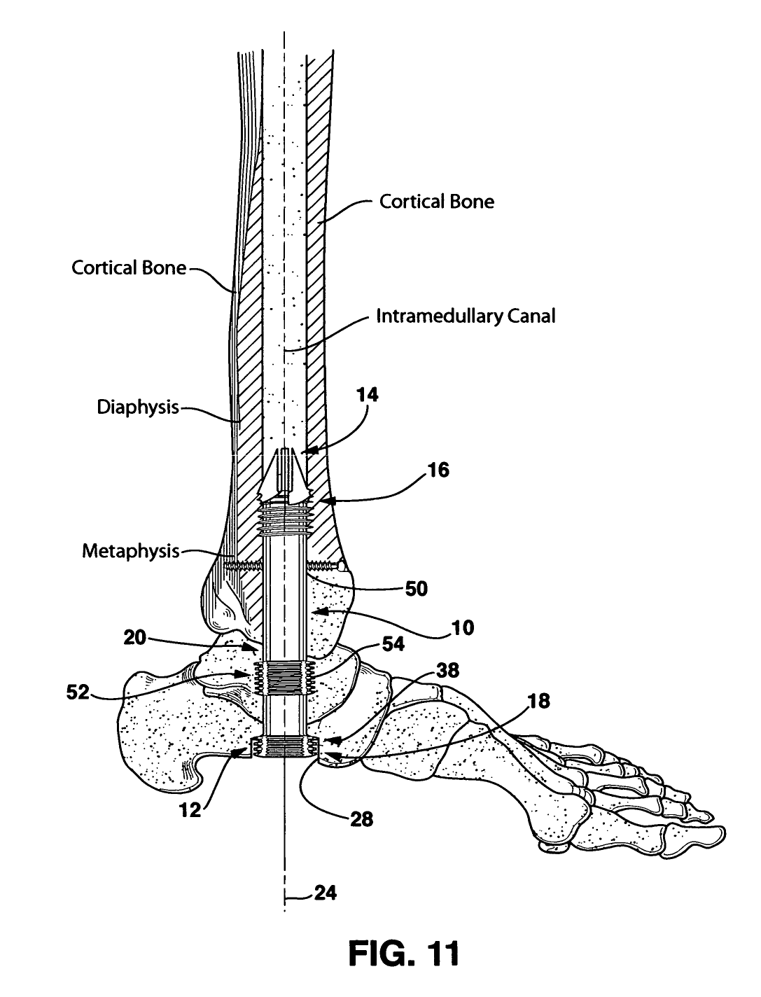

To further lock the ankle fusion device 10 in the bone, as is shown in FIG. 5 in cross-section and in FIGS. 8 and 11, preferably at least one bone screw is placed through the slots 50. These bone screws are preferably hollow bone screws of appropriate length placed over a guide wire, for example a unicortical locked screw or a bicortical screw or other bone screw well understood in the art according to the surgeon's preference.

The distal slots 50 are preferably located by x-ray (fluoroscopy). Where hollow bone screws are used, guide wires are then placed through the bone and through the distal slots 50 going from anterior (the front side) to posterior (the back side). The hollow bone screws are placed on each guide wire and the bone screws screwed into an orientation in the bone passing through a distal slot 50. Interaction between the distal slot 50 and bone screw will prevent ankle fusion device 10 from rotating further and will thereby help to secure ankle fusion device 10 in position in the ankle.

In the alternative or in addition, a bone screw may be placed through the proximal slot 42 at the proximal end 12 of the ankle fusion device 10 as shown in FIG. 5, through the use of guide wires as described above or without the use of guide wires, so that the bone screw will move into contact with and be secured into the bone along the borehole 28 distal to the proximal threaded portion 38. In this way, bone screws help to hold ankle fusion device 10 in place and prevent the ankle fusion device 10 from rotating.

As mentioned above, the proximal slot 42 and the distal slot 50 are preferably oriented at 90 degrees to each other. This allows for optimal location of the locking bone screws through the proximal slot 42 and distal slot 50 into the surrounding bone. Although this is the preferred orientation of the proximal slot 42 and distal slot 50, other orientations may also be used including, but not limited to, the proximal slot 42 and distal slot 50 being aligned and the proximal slot 42 and distal slot 50 being oriented at angles other than 90 degrees.

In the embodiment of the ankle fusion device 10 shown in FIG. 8, the distal threaded portion 32 is placed in the borehole 28 as described above and the ankle fusion device 10 rotated by the interaction of the male tool with tool slot 40 until the middle threaded portion 52 is brought in to contact with the borehole 28. Depending on the pitches P1, P3 of the screw threads of the distal screw thread 36 and the middle screw thread 54, the rotation of the ankle fusion device 10 will cause the distal screw thread 36 to move faster through the borehole 28, slower through the borehole 28 or at the same speed to the borehole 28 as the middle screw thread 54. Where either the distal screw thread 36 moves faster or slower through the borehole 28 than the middle screw thread 54, compressive forces will be applied to the bones through which the distal screw thread 36 and the middle screw thread 54 are located.

Further rotation of the ankle fusion device 10 will ultimately cause the proximal screw thread 44 of the proximal threaded portion 38 to move into contact with the borehole 28. Then, depending on the relationship between the pitch P2 and P1 and P3, further rotation of the ankle fusion device can will cause either the proximal screw thread 44 to want to move faster through the borehole 28, slower through the borehole 28 or at the same speed through the borehole 28 as either or both of the distal screw thread 36 or the middle screw thread 54. Where either the proximal screw thread 44 moves faster or slower through the borehole 28 with respect to the distal screw thread 36 or the middle screw thread 54, compressive forces will be applied to the bones in which the relative cutting portions (36, 44, 54) find themselves in so that compressive pressure is put on the bones to aid in the fusion process.

Using the method and ankle fusion device 10 described above, rigid fixation is immediate and coaption very precise. Autogeneous bone grafting is generally preferred for complex cases although not required. The wounds are closed and the leg placed in a well padded short leg cast. Weight bearing as tolerated is allowed immediately using crutches or a walker.

The ankle fusion device 10 of the present invention in all the different embodiments provides a strong compressive force on the bones of the ankle and is strong enough to endure the stresses and strains placed on the ankle by the patient in the act of walking. Therefore, once the ankle fusion device 10 has been correctly located in the ankle and the fusion process begun, the patient may begin walking on the ankle now containing the ankle fusion device 10 immediately. Experience has shown that the fusion process is completed faster and more effectively if the patient begins walking relatively soon after the ankle fusion device 10 is placed in the ankle to begin the fusion process.

It is anticipated that the ankle fusion device 10 will remain in place in the ankle even after the fusion process is finished. There should be no adverse affect on the patient by leaving the ankle fusion device 10 in place. Once the ankle is immobilized by the ankle fusion device 10 as described above, the bones that will fuse and ultimately form one bone. As a result, the now-fused bones will not move with respect to each other thereby relieving pain from movement of the bones in the former joint. Never-the-less, the ankle fusion device 10 can be easily removed by approaching the calcaneus through the sole incision and using the male member (e.g., screwdriver) to “derotate” the ankle fusion device 10 after the locking screws placed in the proximal slot 42 and distal slot 50 are removed allowing removal of the ankle fusion device 10. After completion of this surgery, the patient is allowed to go home the same day.

In addition to the surgical method described above, a process for improving the outcome of an ankle fusion procedure called “preoperative templating” is preferably used. This templating process means using images such as x-ray images preoperatively to evaluate the size of the bones in the ankle and the ankle itself, match up the sizes of the components of the implant (e.g., ankle fusion device 10 or any other ankle fusion device) with each patient's unique anatomy and then plan the surgical process. This templating process is used since each patient's ankle size and shape will be somewhat unique requiring differently sized implants (e.g., ankle fusion device 10) and individual components.

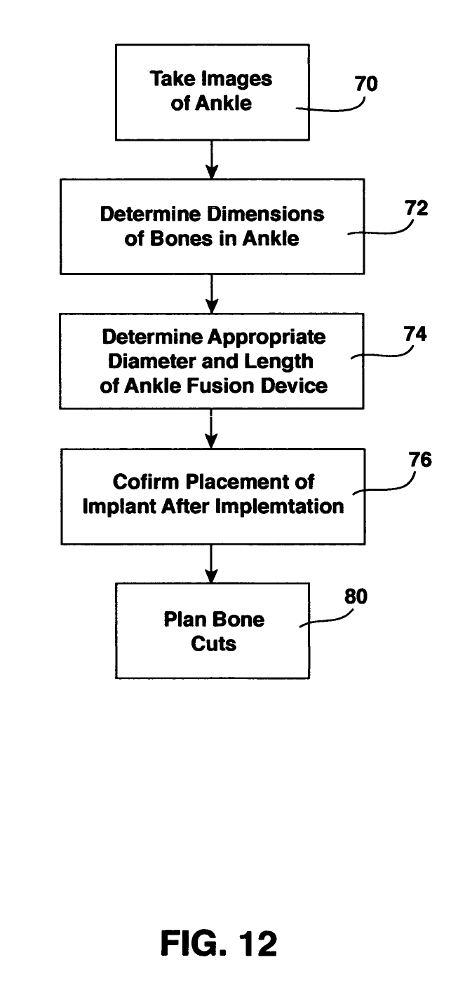

According to this method, shown in a flow chart in FIG. 12, x-ray or other images are taken of the ankle preferably using mortise (bottom of the foot), anterior-posterior (front to back), lateral (side) and oblique (approximately 45 degree) views in conjunction with an index such as a measuring scale (70). Although x-ray images, plain and fluoroscopic, are most commonly used, other images can be used, including without limitation, Magnetic Resonance Imaging (MRI), Computed Axial Tomography (CAT), Positron Emission Tomography (PET), photoacoustic imaging, and ultrasound. Of the types of views typically taken mentioned above, the mortise and the lateral views are usually the most essential views. The oblique view of the ankle will give the practitioner information about the presence of any bone abnormality present preoperatively. The image of the entire tibia and fibula is also preferably taken. Any deformity of the leg that does not allow the foot to be plantigrade in walking must be addressed before or at the time of fusion.

The practitioner then uses these images to determine the dimensions of the ankle and the relevant bones (72). In step 72 the templated mortise x-ray image will show the dimensions of the ankle in total and also of the various bones of the ankle. For example, the x-ray image will show the individual width of the malleoli, the individual width of the distal tibia, the height of the metaphysis (portion of the tibia between the ends) to identify the diaphyseal-metaphyseal junction (the junction of the tibia and fibula with the ankle) and the dimensions of the talus.

After the dimensions of the ankle and the bones of the ankle have been established in step 72, the practitioner uses this information, particularly the tibial dimensions, in step 74 to determine the appropriate size of the ankle fusion device 10 preoperatively. In this step 74, the lateral x-ray image of the ankle from step 72 is particularly helpful to determine the appropriate length and diameter of the ankle fusion device 10.

This step 74 determines the maximum allowable thickness of the ankle fusion device 10 for, particularly, the diaphysis of the tibia. Determining the location of the diaphyseal-metaphyseal junction confirms that an ankle fusion device 10 of proper length is chosen. This step, step 74, is probably the most important step because it will have the biggest effect on the effectiveness of the ankle fusion. After step 74, the method passes to step 76.

Step 76 confirms that sufficient bone will remain after the implant of the ankle fusion device 10 to allow weight bearing immediately after the surgical procedure and thereafter. This step may be done in an iterative process with step 74 so that ultimately the ideal sized ankle fusion device 10 is selected. The method then passes to step 80.

Step 80 plans the precise bone cuts and other key aspects of the surgical procedure to implant the ankle fusion device 10 selected in step 74. At this point most potential surgical issues or problems will have been identified and either dealt with or planned for in this step 80. The surgical procedure starts by planning the approach. The approach may be transfibular, anterior or medially by osteotomizing the medial malleolus.

The advantages of using preoperative templating is that it will naturally hasten the performance of the procedure since potential problems will have been identified in advance and appropriate resolution of such problems planned for. As a result, using this templating method should lessen the complications associated with the ankle fusion procedure in general and also speed up the procedure.

While the templating method has been described in connection with the use of the ankle fusion device 10 in an ankle fusion procedure, the templating method can also be used with any other ankle implant device or in any other ankle procedure.

While the above description contains many specificities, these should not be construed as limitations on the scope of the invention, but rather as examples of preferred embodiments thereof. As a result, the description contained herein is intended to be illustrative and not exhaustive. Many variations and alternatives of the described technique and method will occur to one of ordinary skill in this art. Variations in form to the component pieces described and shown in the drawings may be made as will occur to those skilled in the art. Further, although certain embodiments of an ankle fusion system 10 have been described, it is also within the scope of the invention to add other additional components or to remove certain components such as the distal threaded portion 32, proximal threaded portion 38 or bone screws. Also, variations in the shape or relative dimensions of the tibial component 16, calcaneus component 18, midsection 20, proximal slot 42, distal slot 50, middle threaded portion 52 and bone screws will occur to those skilled in the art and still be within the scope of the invention.

All these alternatives and variations are intended to be included within the scope of the attached claims. Those familiar with the art may recognize other equivalents to the specific embodiments described herein which equivalents are also intended to be encompassed by the claims attached hereto. As a result, while the above description contains may specificities, these should not be construed as limitations on the scope of the invention but rather as examples of different embodiments thereof.

What I claim is:

1. An ankle fusion device comprising:
a cannula having a proximal end, a distal end, a midsection extending between the proximal end and the distal end, a midline and a lumen having an inner surface, the lumen extending along the midline from the proximal end to the distal end;
a tibial component located at the distal end of the cannula wherein the tibial component includes a boring fixture and a distal threaded portion; and
a calcaneus component at the proximal end wherein the calcaneus component includes a proximal threaded portion.

2. The ankle fusion device of claim 1 further wherein the midsection also contains at least one distal slot that extends entirely through the midsection from one side, through the lumen and out the other side of the midsection dimensioned to allow a bone screw to be placed through the distal slot for screw fixation of the ankle fusion device to bone.

3. The ankle fusion device of claim 2 wherein the ankle fusion device has more than one distal slot placed along the length of the midsection either proximally or distally to the original distal slot.

4. The ankle fusion device of claim 1 wherein the boring fixture consists of one or more cutting blades located on the ultimate distal end of the ankle fusion device.

5. The ankle fusion device of claim 4 wherein the boring fixture consists of at least one sharpened blade at the distal end of the cannula

6. The ankle fusion device of claim 5 wherein the at least one sharpened blade curves inward toward the midline as the boring fixture moves toward the distal end of the cannula.

7. The ankle fusion device of claim 1 wherein the distal threaded portion is located proximal to the boring fixture and is a distal screw thread having an outer diameter D1, a core diameter C1 and a constant pitch P1.

8. The ankle fusion device of claim 7 wherein the proximal threaded portion of the calcaneus component is a proximal screw thread having an outer diameter D2, a core diameter C2 and a constant pitch P2.

9. The ankle fusion device of claim 8 wherein pairs of characteristics of the distal threaded portion and proximal threaded portion are formed, each pair formed by the relationships, respectively, between outer diameters D1 and D2, core diameters C1 and C2 and constant pitches P1 and P2, wherein one element of at least one such pair is different from the other element of such pair.

10. The ankle fusion device of claim 8 further comprising a middle threaded portion located along the midsection between the tibial component and the calcaneus wherein the middle threaded portion is a middle screw thread having an outer diameter D3, a core diameter C3 and a constant pitch P3.

11. The ankle fusion device of claim 10 wherein pairs of characteristics of the distal threaded portion, proximal threaded portion and middle threaded portion are formed, each pair formed by the relationships, respectively, between outer diameters D1, D2 and D3, core diameters C1, C2 and C3 and constant pitches P1, P2 and P3, wherein one element of at least one such pair is different from the other element of such pair.

12. The ankle fusion device of claim 1 wherein the proximal threaded portion of the calcaneus component is a proximal screw thread having an outer diameter D2, a core diameter C2 and a constant pitch P2.

13. The ankle fusion device of claim 1 wherein the calcaneus component further includes a tool slot and a proximal slot.

14. The ankle fusion device of claim 13 wherein the tool slot is a cavity formed in the proximal end of the ankle fusion device.

15. The ankle fusion device of claim 14 wherein the cavity of the tool slot has a shape that conforms with the external configuration of a male tool that conformally mates with the tool slot.

16. The ankle fusion device of claim 15 wherein the tool slot has a shape chosen to mate a male tool having a shape chosen from the group consisting of hexagonal, square, triangular, pentagonal or star.

17. The ankle fusion device of claim 1 wherein the calcaneus component includes a proximal slot extending from the lumen at an angle outward through the proximal threaded portion to the outer surface of the proximal threaded portion.

18. The ankle fusion device of claim 17 wherein the proximal slot is a slot having a diameter larger than the diameter of a guide wire is used to position the ankle fusion device.

19. The ankle fusion device of claim 17 wherein the proximal slot extends from the lumen at an angle outward through the proximal threaded portion to the outer surface of the proximal threaded portion.

20. The ankle fusion device of claim 17 wherein the proximal slot also extends distally from the lumen through the wall of the midsection inline with the part of the proximal slot that extends proximally from the lumen.

21. The ankle fusion device of claim 1 wherein the tibial component, calcaneus component and midsection are molded in one piece of a rugged, durable, biocompatible material.

22. The ankle fusion device of claim 1 wherein the tibial component, calcaneus component and midsection are manufactured separately of the same or different material and joined together.

23. The ankle fusion device of claim 1 wherein the surface of the midsection is studded to enhance fixation to the surrounding bone.

24. The ankle fusion device of claim 1 wherein the surface of the midsection is sintered to enhance fixation to the surrounding bone.

25. The ankle fusion device of claim 1 wherein the outer surface of the midsection has an osteoconductive coating.

26. The ankle fusion device of claim 1 wherein the lumen has a diameter capable of receiving a guide wire or guide pin into the lumen from the distal end toward the proximal end.

27. An ankle fusion device comprising:
a cannula having a proximal end, a distal end, a midsection extending between the proximal end and the distal end, a midline and a lumen extending along the midline from the proximal end to the distal end wherein the midsection also contains at least one distal slot that extends entirely through the midsection from one side, through the lumen and out the other side of the midsection dimensioned to allow a bone screw to be placed through the distal slot for screw fixation of the ankle fusion device to the bone;
a tibial component located at the distal end of the cannula wherein the tibial component includes a distal threaded portion located at the distal end of the cannula and being a distal screw thread and having an outer diameter D1, a core diameter C1 and a constant pitch P1 wherein the tibial component also includes a boring fixture consisting of one or more cutting blades located on the ultimate distal end of the ankle fusion device; and
a calcaneus component at the proximal end of the cannula wherein the calcaneus component includes a proximal threaded portion located at the proximal end and being a proximal screw thread having an outer diameter D2, a core diameter C2 and a constant pitch P2; and
a middle threaded portion located along the midsection between the tibial component and the calcaneus component wherein the middle threaded portion is a middle screw thread having an outer diameter D3, a core diameter C3 and a constant pitch P3;
wherein pairs of characteristics of the distal threaded portion and proximal threaded portion are formed, each pair formed by the relationships, respectively, between outer diameters D1 and D2, core diameters C1 and C2 and constant pitches P1 and P2, wherein one element of at least one such pair is different from the other element of such pair.

28. A method for improving the outcome of an ankle fusion procedure consisting of the steps of:
(a) obtaining at least one image of the ankle;
(b) determining the dimensions of the relevant bones of the ankle and of the ankle itself using the image or images from step (a);
(c) determining the appropriate dimensions of an implantable device using the image or images and dimensions from steps (a) and (b);
(d) determining whether there will be sufficient bone remaining after implant of the implantable device of step (c) to form a strong ankle fusion;
(e) planning an ankle fusion procedure using the information from steps (b) and (c).
